(19) United States
(12) Patent Application Publication
(10) Pub. No.: US 2026/0204033 A1
(43) Pub. Date: Jul. 16, 2026

(54) AI-ENHANCED DEPTH PERCEPTION AND ANATOMICAL HIGHLIGHTING SYSTEM FOR VIDEO-ASSISTED MEDICAL PROCEDURES

(71) Applicant: Intublade Co., Odessa, FL (US)

(72) Inventor: Andrew B. Napier, Odessa, FL (US)

(73) Assignee: Intublade Co., Odessa, FL (US)

(21) Appl. No.: 19/017,916

(22) Filed: Jan. 13, 2025

(51) Int. Cl. G06T 19/20; A61B 34/00; G06T 7/50; G06T 15/20; G06V 10/70; G16H 10/60; G16H 20/40

(52) U.S. Cl. CPC G06T 19/20; A61B 34/25; G06T 7/50; G06T 15/20; G06V 10/70; G16H 10/60; G16H 20/40; G06T 2200/24; G06T 2207/10016; G06T 2207/10068; G06T 2207/20081; G06T 2207/30004; G06T 2210/41; G06T 2210/62; G06T 2219/004; G06T 2219/2012; G06V 2201/03

(57) ABSTRACT

The disclosure relates to an AI-enhanced system for video-assisted medical procedures. It offers enhanced depth perception and real-time anatomical highlighting, integrating AI algorithms with existing medical imaging systems. The system includes continuous learning, broad applicability across various procedures, a user-friendly interface, and cloud-based updates, significantly enhancing medical procedural safety, accuracy, and efficiency.

### CROSS-REFERENCE TO RELATED APPLICATION

This application claims priority to U.S. Provisional Ser. No. 63/619,785, filed Jan. 11, 2024, the contents of which are hereby incorporated in their entirety as if fully set forth herein.

### FIELD OF THE DISCLOSURE

The present disclosure is directed to medical imaging technology and artificial intelligence (AI), specifically focusing on enhancing the capabilities of video-assisted medical procedures.

### BACKGROUND

In the realm of medical procedures, particularly in procedures involving internal visualization of the body, the reliance on video feeds is paramount. Traditional systems provide two-dimensional views, which pose significant limitations in terms of depth perception and clarity in identifying crucial anatomical structures. This often results in a higher risk of procedural errors, extended operation times, and increased difficulty for practitioners, particularly those in training or with less experience.

Current solutions to these challenges primarily revolve around hardware upgrades, such as the introduction of additional optical components or switching to more sophisticated camera systems like stereo or 3D cameras. However, these solutions come with significant drawbacks, including high costs, increased complexity, and the need for extensive retraining of medical personnel. Moreover, these hardware-focused solutions are not always universally adaptable to different types of medical procedures or equipment, limiting their applicability.

Moreover, existing software solutions that attempt to enhance video feeds lack the dynamic adaptability and precision required for complex medical scenarios. They often fail to provide the depth of analysis and real-time feedback necessary for high-stakes medical environments. As such, there remains a significant gap in technology that can effectively address these challenges in a cost-effective, universally adaptable, and easily deployable manner.

Thus, there is a pressing need for innovative solution, including systems, device and methods, that can enhance the capabilities of existing video-assisted medical devices.

### SUMMARY OF THE DISCLOSURE

In certain embodiments, the method for enhancing depth perception in medical procedures utilizes a procedural element with an integrated high-resolution camera for capturing monocular video feeds within the human body. This element, adaptable to various medical devices like endoscopes or catheters, captures detailed imagery of internal anatomy. The video feed is transmitted in real-time to an external system equipped with advanced computing capabilities, including a high-speed processor and extensive memory.

The external system processes the monocular video feed using sophisticated AI algorithms. These algorithms analyze the feed to extract depth cues, applying machine learning and computer vision techniques such as pattern recognition and edge detection. The result is a dynamic, real-time, three-dimensional perspective of the internal view, presented to the practitioner through an interactive interface.

This method offers a significant enhancement over traditional monocular views in medical imaging, providing a more comprehensive understanding of spatial relationships in internal structures. It is particularly beneficial for precision-required procedures, where understanding the depth and spatial arrangement of tissues is crucial. The system's AI capabilities also extend to recognizing and highlighting specific anatomical features and pathologies, thus aiding in accurate diagnosis and treatment. This innovative approach marks a considerable advancement in medical imaging, potentially improving procedural accuracy and patient outcomes.

### BRIEF DESCRIPTION OF THE DRAWINGS

A clear understanding of the key features of the AI-Enhanced Depth Perception and Anatomical Highlighting System summarized above may be had by reference to the appended drawings, which illustrate the method and system of the disclosure. It is to be understood that such drawings depict preferred embodiments of the disclosure and are not to be considered as limiting its scope.

FIG. 1 is a schematic block diagram of one embodiment of a medical system, according to the disclosure;

FIG. 2 is a schematic block diagram of another embodiment of a medical system, according to the disclosure;

FIG. 3A is a schematic block diagram of another embodiment of a medical system, according to the disclosure;

FIG. 3B is a schematic block diagram of an arrangement in which multiple devices can access a database (or store) for uploading and downloading information, according to the disclosure;

FIG. 4 is a schematic block diagram of one embodiment of an interface unit, according to the disclosure;

FIG. 5 is a schematic block diagram of one embodiment of an external processing unit, according to the disclosure;

FIG. 6 is a schematic block diagram of one embodiment of a database, according to the disclosure;

FIG. 7A-B are illustrations of AI-Enhanced depth perception and anatomical highlighting system;

FIG. 8 is a flowchart illustrating the method of depth perception enhancement in the AI processing unit, detailing the process from capturing the video feed to displaying the enhanced feed with added depth cues;

FIG. 9 is a flowchart illustrating the process of real-time anatomical highlighting, showing the sequence from inputting the video feed to highlighting key anatomical structures within the feed;

FIG. 10 is a user interface mockup, presenting a simplified representation of the display interface with the enhanced video feed, depth cues, and anatomical highlights, alongside the interactive elements like buttons and sliders for customization;

FIG. 11 is a diagram illustrating the continuous learning and improvement cycle, showing the integration of diverse data sources, feedback mechanisms, and periodic AI model updates;

FIG. 12 is a schematic of the system's integration within a typical medical setting, depicting the connections between the medical imaging device, AI processing unit, and cloud-based services;

FIG. 13 is a detailed illustration of the cloud-based model training and update facility, showcasing how data is collected, processed, and utilized to enhance the AI model continuously; and

FIG. 14 is a diagram of the broad applicability of the system across various medical procedures, demonstrating the system's adaptability to different types of medical devices and procedural environments.

These figures are intended to provide a visual representation of the various aspects and functionalities of the medical system, emphasizing its versatility, user-friendliness, and innovative integration of AI into medical imaging technology.

### DETAILED DESCRIPTION OF THE DISCLOSURE

As used herein, the term “proximal,” when used in connection with a device or system, refers to a position relatively close to the user of that device or system when it is being used as intended, while the term “distal” refers to a position relatively far from the user of the device. In other words, the leading end of a device or system is positioned distal to the trailing end of the delivery device or system, when the device is being used as intended. As used herein, the terms “substantially,” “generally,” “approximately,” and “about” are intended to mean that slight deviations from absolute are included within the scope of the term so modified.

The present disclosure is directed to advancements in medical imaging technology and artificial intelligence (AI), specifically focusing on enhancing the capabilities of video-assisted medical procedures. It encompasses methods, systems, and devices designed to provide AI-driven assistance through real-time machine learning applications, including real-time anatomical tagging, real-time guidance systems, and assimilated scenario testing to aid clinicians during critical medical interventions.

In various medical procedures, particularly those involving internal visualization such as endoscopy, bronchoscopy, cystoscopy, and video laryngoscopy, clinicians rely heavily on video feeds to navigate and perform precise interventions. Traditional video-assisted systems provide two-dimensional (2D) views, which inherently limit clarity in identifying crucial anatomical structures. This limitation can lead to increased procedural errors, prolonged operation times, and heightened difficulty for practitioners, especially those with less experience or those undergoing training.

As previous noted, existing solutions to enhance video-assisted medical procedures predominantly focus on hardware improvements. These include integrating additional optical components or transitioning to more sophisticated camera systems, such as stereo or three-dimensional (3D) cameras. However, these hardware-centric solutions often entail significant drawbacks, including elevated costs, increased system complexity, and the necessity for extensive retraining of medical personnel. Furthermore, such hardware enhancements are not universally adaptable across different types of medical procedures or equipment, thereby restricting their broader applicability.

Conversely, current software solutions attempting to enhance video feeds fall short in providing the dynamic adaptability and precision required for intricate medical scenarios. These software enhancements frequently lack the depth of analysis and real-time feedback necessary for high-stakes medical environments. Consequently, there exists a substantial gap in technology that can effectively address these challenges in a cost-effective, universally adaptable, and easily deployable manner.

Therefore, there is an urgent need for innovative systems, devices, and methods that can significantly enhance the capabilities of existing video-assisted medical devices. These enhancements should focus on providing AI-driven assistance through real-time machine learning applications, including real-time anatomical tagging, real-time guidance systems, and assimilated scenario testing to facilitate more accurate and efficient medical procedures. Such a solution should not only improve the safety and efficiency of these procedures but also be easily integrated into existing medical workflows without the need for extensive hardware modifications or overhauls. This disclosure aims to meet these needs by providing an AI-enhanced system for depth perception and anatomical highlighting, adaptable across various medical specialties and procedures. It also addresses the need for improved depth perception and real-time anatomical highlighting in various procedures including endoscopy, bronchoscopy, cystoscopy, and video laryngoscopy. The disclosure also provides a novel approach to augmenting standard monocular camera feeds used in these procedures with advanced AI-driven analysis.

Thus, the present disclosure introduces an AI-driven system designed to augment video-assisted medical procedures by leveraging real-time machine learning applications. This system may integrate advanced AI algorithms with existing medical imaging technologies to provide clinicians with enhanced visual information, thereby improving procedural accuracy, safety, and efficiency. The system may include three primary AI-driven applications: real-time anatomical tagging, real-time guidance systems, and assimilated scenario testing, while also incorporating robust data management and continuous learning capabilities.

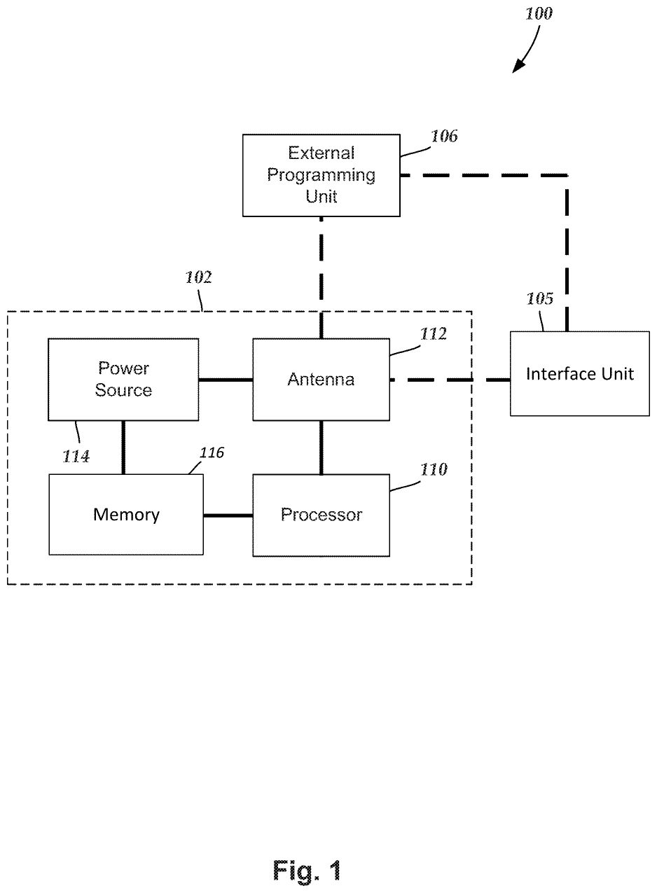

FIG. 1 illustrates schematically one embodiment of a medical system 100 that includes a procedural element (e.g., a laryngoscope) 102, one or more external processing units 106, and an interface unit 105. Although FIG. 1 illustrates one external processing units 106, one procedural element 102, and one interface unit 105, it will be understood that the system can include more than one external processing unit, more than one control module, and more than one interface unit. Additionally, interface unit 105 and external processing unit 106 may be the same device. The procedural element 102 may include a processor 110, an antenna 112 (or other communications arrangement), a power source 114, and a memory 116, as illustrated in FIG. 1. In some examples, processor 110 of procedural element 102 may manage the capture and initial processing of video feeds and then send the information external systems (e.g., external processing unit 106) for further analysis. External processing unit 106 may be responsible for advanced data analysis and AI-driven enhancements and (when combined with interface unit 105), may provide an interactive interface that presents enhanced video feeds, anatomical tagging, and guidance feedback to the clinician, supporting customization and real-time adjustments based on procedural needs.

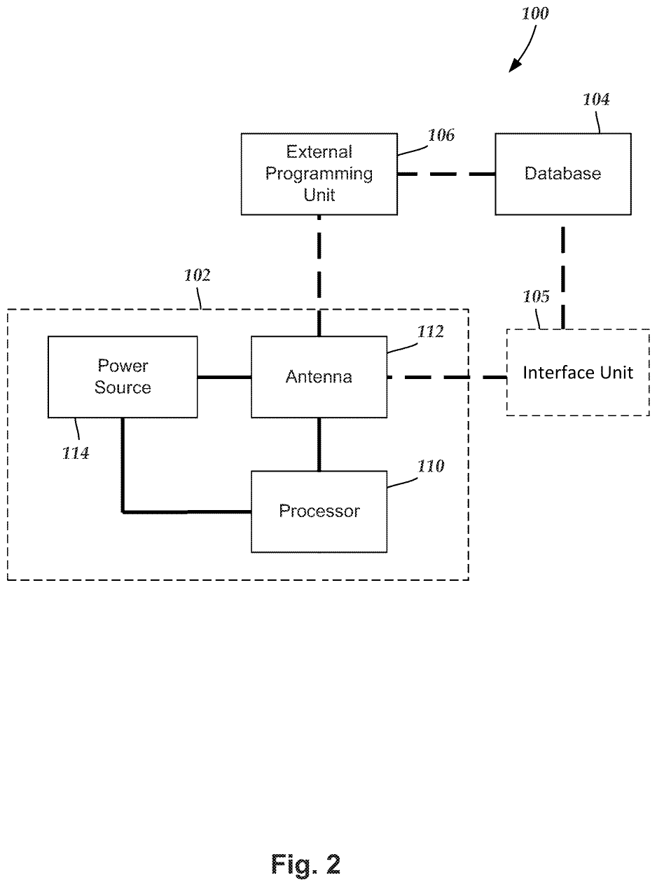

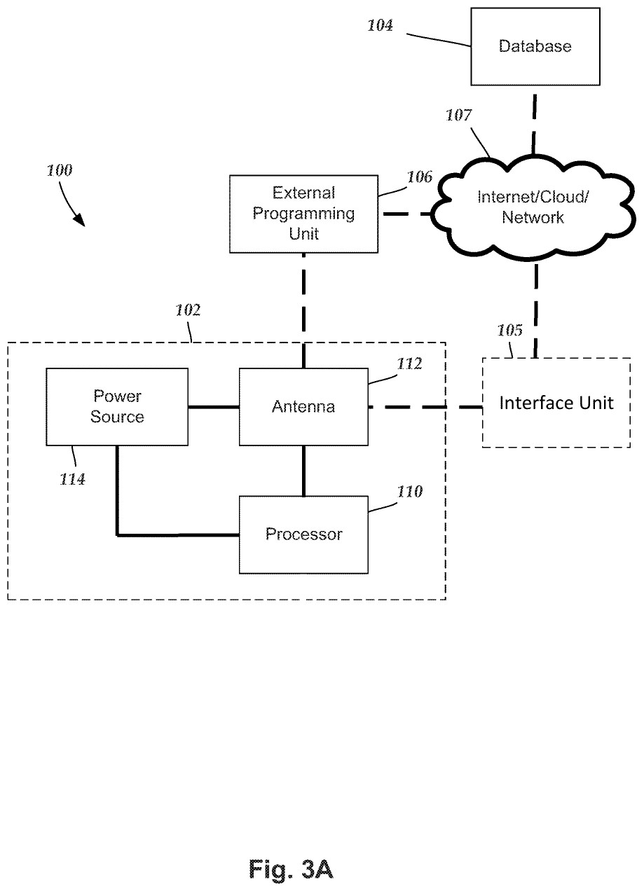

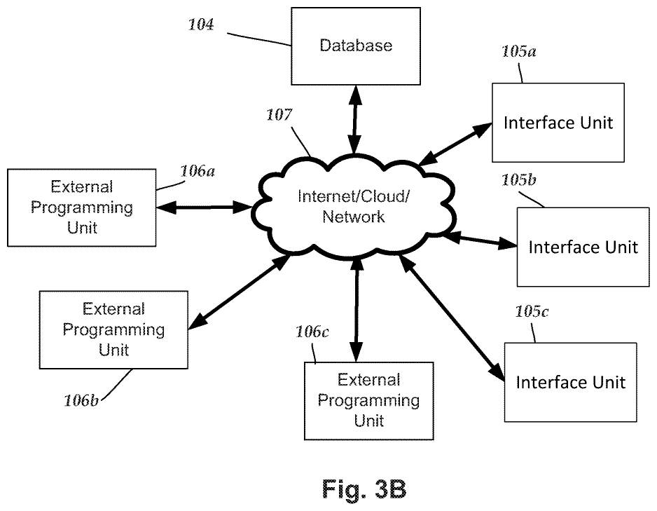

FIG. 2 illustrates another embodiment that includes a database 104 which communicates with the external processing unit 106 and optional interface unit 105. FIG. 3A illustrates another embodiment in which the external processing unit 106 and optional interface unit 105 communicate through the Internet, a cloud, or a local or wide area network 107 (including wireless local or wide area networks) or any combination thereof, or any other suitable intermediary or combination of intermediaries, to the database 104.

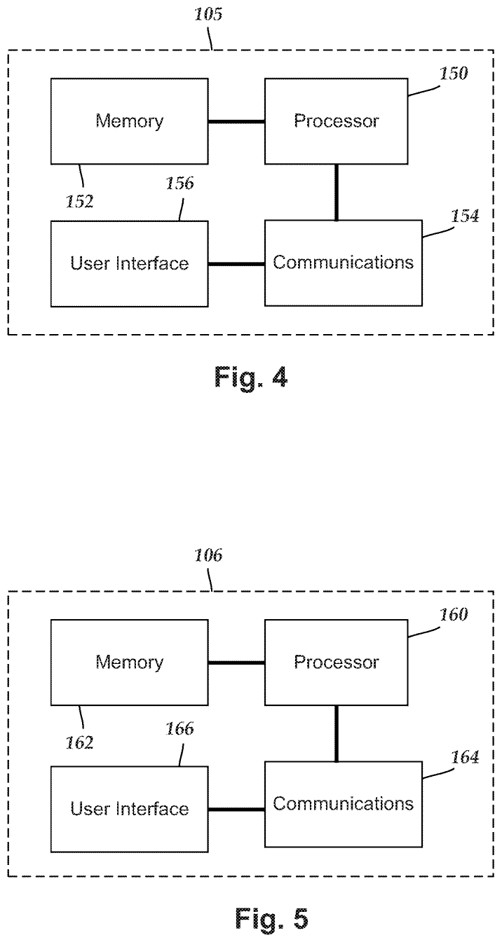

One example of an interface unit 105 is illustrated in FIG. 4 and includes a processor 150, a memory 152, a communications arrangement 154 (such as an antenna, USB-C cable, or any other suitable communications device such as those described below), and an optional user interface 156. Suitable devices for use as an interface unit can include, but are not limited to, a computer, a tablet, a mobile telephone, a personal desk assistant, a dedicated device for external programming, remote control, or the like. In at least some embodiments, the interface unit 105 can also be an external processing unit.

One example of an external processing unit 106 is illustrated in FIG. 5 and includes a processor 160, a memory 162, a communications arrangement 164 (such as an antenna or any other suitable communications device such as those described below), and a user interface 166. Suitable devices for use as an external processing unit can include, but are not limited to, a computer, a tablet, a mobile telephone, a personal desk assistant, a dedicated device for external programming, remote control, or the like. It will be understood that the external processing unit 106 and interface unit 105 can include a power supply or receive power from an external source or any combination thereof. In at least some embodiments, the external processing unit 106 may also be an interface unit.

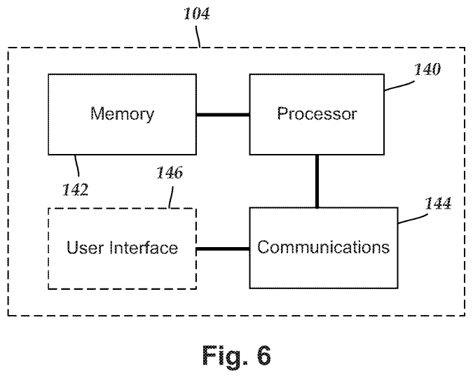

One example of a database 104 is illustrated in FIG. 6 and includes a processor 140, a memory 142, a communications arrangement 144 (such as an antenna or any other suitable communications device such as those described below), and an optional user interface 146. Suitable devices for use as a remote data storage unit can include, but are not limited to, a computer, a tablet, a server or server farm, a dedicated device for data storage, a hard drive, cloud storage arrangement, or the like. It will be understood that the external processing unit 106 and remote data storage unit 104 can include a power supply or receive power from an external source or any combination thereof. In some embodiments, the database 104 can also act as a remote storage unit for storage and retrieval of procedure parameters and other procedure data.

Methods of communication between devices or components of a system can include wired or wireless (e.g., RF, optical, infrared, near field communication (NFC), Bluetooth™, or the like) communications methods or any combination thereof. By way of further example, communication methods can be performed using any type of communication media or any combination of communication media including, but not limited to, wired media such as twisted pair, coaxial cable, fiber optics, wave guides, and other wired media and wireless media such as acoustic, RF, optical, infrared, NFC, Bluetooth™ and other wireless media. These communication media can be used for communications units 144, 154, 164 or as antenna 112 or as an alternative or supplement to antenna 112.

Turning to the procedural element 102, some of the components (for example, a power source 114, an antenna 112, and a processor 110) of the system can be positioned on one or more circuit boards or similar carriers within a sealed housing of the procedural element 102, if desired. Any power source 114 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like.

If the power source 114 is a rechargeable battery, the battery may be recharged using the antenna 112, if desired. Power can be provided to the battery for recharging by inductively coupling the battery through the antenna to a recharging unit.

With respect to the procedural element 102, interface unit 105, external processing unit 106, and database unit 104, any suitable processor 110, 140, 150, 160 can be used in these devices. For the procedural element 102, the processor 110 is capable of receiving and interpreting instructions from an external processing unit 106 that, for example, allows modification of imaging characteristics. In the illustrated embodiment, the processor 110 is coupled to the antenna 112. This allows the processor 110 to receive instructions from the external processing unit 106, if desired. The antenna 112, or any other antenna described herein, can have any suitable configuration including, but not limited to, a coil, looped, or loopless configuration, or the like.

In one embodiment, the antenna 112 is capable of receiving signals (e.g., RF signals) from the external processing unit 106. The external processing unit 106 can be a home station or unit at a clinician's office or any other suitable device. The external processing unit 106 can be any unit that can provide information to the procedural element 102. One example of a suitable external processing unit 106 is a computer operated by the user or clinician to send signals to procedural element 102. Another example is a mobile device or an application on a mobile device that can send signals to procedural element 102.

The signals sent to the processor 110 via the antenna 112 can be used to modify or otherwise direct the operation of the procedural element. For example, the signals may be used to modify the resolution, contrast, brightness, exposure, or location of the images taken. The signals may also direct the procedural element 102 to cease operation, to start operation, to start charging the battery, or to stop charging the battery.

Optionally, procedural element 102 may include a transmitter (not shown) coupled to the processor 110 and the antenna 112 for transmitting signals back to the external processing unit 106 or another unit capable of receiving the signals. For example, the control module 102 may transmit signals indicating whether the procedural element 102 is operating properly or not or indicating when the battery needs to be charged or the level of charge remaining in the battery. The processor 110 may also be capable of transmitting information about the imaging characteristics so that a user or clinician can determine or verify the characteristics.

Any suitable memory 116, 142, 152, 162 can be used for the respective components of the system 100. The memory 116, 142, 152, 162 illustrates a type of computer-readable media, namely computer-readable storage media. Computer-readable storage media may include, but is not limited to, nonvolatile, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The user interfaces 156, 166 of the external processing unit 106 and interface unit 105 and optional user interface 146 of the database 104 can be, for example, a keyboard, mouse, touch screen, track ball, joystick, voice recognition system, or any combination thereof, and the like. Alternatively, or additionally, the user interfaces 156, 166 of the external processing unit 106 and of the interface unit 105 can include one or more microphones, sensors, cameras, or the like to obtain patient feedback. For example, the patient may provide feedback verbally (e.g., voice command recognition, voice recordings) or visually (e.g., video of patient, non-touch gesture recognition, or the like). In at least some embodiments, at least a portion (or even all of) the patient feedback may be recorded as bio-signals from the patient (EEG, EMG, CMAP, ECG, skin resistance, muscle tone, movement, vibration, rigidity, temperature, breathing, oxygen levels, chemical concentrations, skin resistance, gait, skin tone, force, pressure, or the like.) In at least some embodiments, such as those illustrated in FIGS. 2 and 3 without the optional interface unit, patient feedback can be provided by the clinician or other user through the external processing unit 106.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks or described for the control modules, external processing units, remote data storage units, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the disclosure.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

As indicated above, information from multiple patients can be aggregated in a database 104 and used to provide additional procedural recommendations. FIG. 3B illustrates an arrangement with multiple external processing units 106 a, 106 b, 106c and multiple patient user interfaces 105a, 105b, 105c that can access a database 104 through the Internet, a cloud, or a local or wide area network 107, or any combination thereof. In at least some embodiments, the database 104 can be a repository or store for obtaining procedural programs, procedural parameters, procedural adjustments, therapy recommendation or guidance, or other information for use in diagnosing or treating a patient.

In at least some embodiments, machine learning or other algorithms may be used to analyze the data on the database 104 to identify correlations between procedural efficacy and one or more variables including, but not limited to, disease or disorder or condition being treated; patient demographics (e.g., age, ethnicity, gender, activity level, weight, or the like); physical activity; or the like or any combination thereof. In at least some embodiments, the results of these algorithms may be tailored to a particular group of patients; one or more specific patient characteristics; one or more specific diseases or disorders or conditions; one or more specific symptoms; or the like or any combination thereof.

In at least some embodiments, the database 104 may include previously uploaded programs or sets of parameters; internally or externally generated programs or sets of parameters (generated using, for example, machine learning or other algorithms); improved programs or sets of parameters; or the like or any combination thereof. In at least some embodiments, these programs or sets of parameters (or other information in the database 104) may be downloaded by a patient, clinician, or other person onto a control module 102, external processing unit 106, patient user interface 105, or any other suitable device. In at least some embodiments, downloading of some or all of the information may be limited to specific users (e.g., clinicians or paying subscribers).

In the following examples, the procedural element is a laryngoscope and the disclosure relates to an AI-enhanced depth perception and anatomical highlighting system,“ which introduces a novel system for augmenting video-assisted medical procedures. The system leverages advanced artificial intelligence algorithms to substantially improve depth perception and enable real-time highlighting of anatomical structures from monocular video feeds typical in medical devices like endoscopes, bronchoscopes, laryngoscopes, and similar apparatus.

To recap the system components and architecture at a high level, it will be sufficient to note that the system may include a procedural element, which may be any one or more medical device equipped with a high-resolution monocular camera capable of capturing real-time video feeds from within the body during procedures such as intubation, endoscopy, bronchoscopy, and cystoscopy. The system may also include an external processing (or computing) unit equipped with high-performance processing capabilities and extensive memory, responsible for processing the captured video feeds using sophisticated AI algorithms. This processing unit supports the primary AI-driven applications by providing the necessary computational power for real-time analysis and feedback. AI algorithms (e.g., machine learning and computer vision techniques), may include but are not limited to semantic segmentation, object detection, and predictive analytics, and these may be employed to analyze video feeds. These algorithms may facilitate real-time anatomical tagging, guidance systems, and scenario testing, enhancing the overall procedural support provided to clinicians. An interactive user interface may present the AI-enhanced video feed to clinicians. This interface includes features such as adjustable settings for anatomical tagging and guidance feedback, as well as tools for interacting with assimilated scenario testing modules. The system may also include cloud-based model training and updates, and this may be a scalable cloud infrastructure that facilitates the training and continuous improvement of AI models. This infrastructure allows for the aggregation and processing of extensive procedural data from diverse medical settings, ensuring that AI algorithms remain up-to-date with the latest medical practices and insights. Finally, the system may include edge deployment hardware in the form of specialized hardware deployed within medical environments to enable real-time inference and processing of AI models. This hardware ensures low latency and high accuracy in providing anatomical tagging and guidance feedback during procedures.

Several functionalities may be provided using the system, and the following non-exhaustive list briefly describes these advantages. First, the system may provide real-time anatomical tagging using AI-driven semantic segmentation and object detection to accurately identify and label anatomical structures in real time. This functionality aids clinicians in avoiding critical areas, thereby enhancing precision during medical procedures. Examples of tagged structures include the epiglottis, false cords, cricoid ring, true cords, interarytenoid notch, and other relevant anatomical landmarks.

Second, the system may provide AI-enhanced real-time guidance, which presents dynamic, real-time feedback to clinicians during procedures. This may, for example, assist in maintaining optimal positioning and orientation of the procedural element, reducing procedural errors and enhancing overall efficiency. For example, the system may continuously analyze the procedural environment to predict optimal angles and positions for the procedural element, ensuring precise navigation. The system may also provide dynamic adjustment by refining guidance parameters in real time based on changes in the procedural environment, such as patient movement or anatomical variations, to maintain consistent procedural accuracy. It may also provide a feedback mechanism including visual and/or auditory cues to inform clinicians of necessary adjustments, thereby reducing the likelihood of procedural errors.

Third, the system may provide assimilated scenario testing by integrating augmented reality (AR) simulations to replicate complex procedural scenarios enabling comprehensive testing and validation of AI algorithms. These simulations allow for comprehensive testing and validation of the system's response to challenges such as fluid obstructions, low-light conditions, and anatomical variations, ensuring robustness and reliability before clinical deployment. It may also provide a platform for training clinicians and validating AI-driven assistance mechanisms, enhancing both user proficiency and the system.

Fourth, the system may provide labeling and data management functionalities with an AI-optimized labeling tool to facilitate the accurate and efficient annotation of anatomical structures within video feeds. This tool may leverage a client-server architecture to host pre-trained models, enabling rapid and precise data labeling without the need for high-end local hardware.

Fifth, the system may include continuous learning and adaptation using machine learning techniques and periodic model updates based on new procedural data and clinician feedback. This continuous learning mechanism ensures that AI models adapt to a wide range of medical scenarios and patient profiles, enhancing their accuracy and reliability over time. Finally, the system may provide user interaction and customization by supporting various interaction modes, including touch and voice commands. With this tool, clinicians can customize settings to suit specific procedural requirements, enhancing the system's versatility across different medical environments.

Another central feature of this disclosure is its ability to enhance depth perception in medical imaging. It may achieve this by employing AI algorithms that process the video feeds captured during medical procedures, and interpreting visual cues from the monocular camera feed to construct depth maps. This transformation of two-dimensional images into a more comprehensible three-dimensional perspective is desirable in medical settings where depth perception is vital for the accuracy and safety of various procedures.

Another pivotal aspect of the disclosure is its real-time anatomical highlighting capability. Using machine learning techniques such as semantic segmentation and object detection, the system processes video feeds to identify and visually emphasize essential anatomical structures. This real-time highlighting aids practitioners in swiftly recognizing key areas during medical procedures, thereby improving the accuracy and efficacy of both diagnostics and treatments. In some examples, the system may utilize semantic segmentation and/or object detection algorithms to identify and label key anatomical structures in real time.

The depth perception and/or the anatomical mapping may also be useful when used as part of a guidance module, which employs predictive analytics and machine learning techniques to provide dynamic guidance feedback, assisting clinicians in maintaining optimal procedural element positioning and orientation. For example, a system may calculate and/or predict an optimal position of the procedural element, and may alert the user when the procedural element drifts away from the optimal position by a predetermined amount. In some examples, the alert may be auditory, visual, haptic or some combination thereof.

The disclosure is designed with versatility and adaptability in mind, allowing its application across a range of video-assisted medical procedures. It offers customizable settings to adapt its functionalities to the specific requirements of different medical procedures. This flexibility ensures that the system can be seamlessly integrated into various medical practice environments.

Ease of use and integration is a hallmark of the system's design, featuring a user-friendly interface that presents the AI-enhanced video feed with depth cues and highlighted anatomical features. This interface is designed to be intuitive and accommodating to individual user preferences and procedural needs.

Additionally, a significant advantage of the system is its continuous learning and improvement mechanism. The AI model is designed to evolve by analyzing a diverse range of procedural videos and feedback from medical professionals, ensuring continuous refinement and adaptation to the latest medical practices and insights.

To maintain its technological edge, the system includes a cloud-based facility for AI model training and updates. This feature allows for the processing of extensive datasets and the deployment of regular updates to the AI processing unit, ensuring the system remains at the forefront of medical technology advancements.

Thus, the instant disclosure represents a significant leap forward in the field of medical imaging. By integrating AI-driven depth perception and anatomical highlighting into existing medical imaging technologies, it aims to enhance patient safety, procedural efficiency, and overall medical outcomes across various medical specialties. Specifically, by providing accurate real-time anatomical tagging and guidance, the system may reduce failed intubation attempts, particularly in complex cases. Moreover, instantaneous adjustments may streamline procedural actions, enhancing workflow efficiency during emergencies and reducing procedural time. Furthermore, complications may be reduced as maintaining optimal procedural element positioning reduces trauma risks, enhancing patient safety. AI-driven video processing and AR simulations may enhance training as they offer realistic, hands-on practice for clinicians, improving training efficacy.

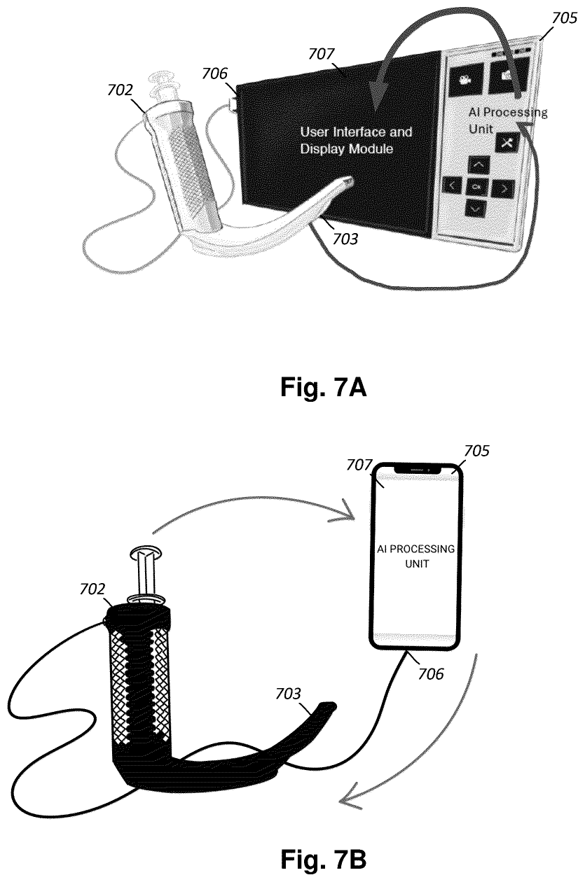

Referring to FIG. 7A-B, examples of AI-enhanced depth perception and anatomical highlighting system 700 are shown. As illustrated, the comprehensive system architecture 700, includes a laryngoscope 702 and an AI processing unit 705. Laryngoscope 702 may be a video laryngoscope including a camera 703, and AI processing unit 705 may include a video input module 706, and a User Interface and display module 707. Laryngoscope 702 and specifically camera 703 may capture images and/or video and transmit them to video input module 706 to provide real-time video feeds of internal anatomical structures. The real-time video and/or image feed may then be fed into the AI processing unit 705 and/or displayed on display module 707 alone or with other information.

An exemplary operational workflow may encompass three primary stages: labeling, training and testing/integration. In the labeling stage, data (e.g., raw video feeds) ma be captured and collected using the procedural element during medical interventions. This data may be labeled by having the collected raw data transmitted to the labeling tool hosted on a server. Here, AI-optimized models may perform rapid and accurate annotation of anatomical structures, enhancing dataset accuracy and reducing labeling time. Alternatively, this data may be manually annotated. The labeled data may then be stored securely in the cloud-based storage system, ready for use in model training. The second stage may be the training stage. This may include model selection where one or more appropriate neural network (NN) models are selected based on the specific requirements of anatomical tagging and guidance systems. The selected NN models are trained using the labeled datasets on cloud-based high-performance computing instances, enabling rapid experimentation and refinement. The system may monitor model performance metrics to ensure optimal accuracy and efficiency, facilitating timely adjustments and improvements. Finally, after the labeling and training stage, the testing/integration stage may be employed. This may include real-world testing where trained models undergo rigorous testing in simulated and controlled environments to validate their performance under diverse medical scenarios. Successfully tested models are deployed to edge hardware within medical settings, ensuring real-time inference capabilities during actual procedures, and the AI-driven system may be integrated into existing medical workflows, providing clinicians with enhanced visual tools to improve procedural outcomes.

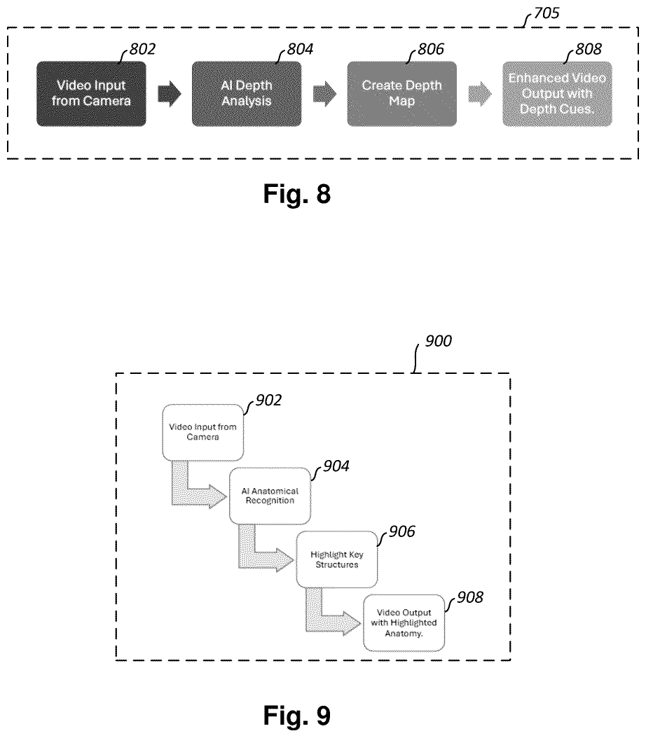

Turning to FIG. 8, a flowchart demonstrates the depth perception enhancement process within AI processing unit 705. This chart delineates the sequence from capturing video feed 802 to AI depth analysis 804, followed by creating a depth map 806 and displaying the enhanced feed 808, which incorporates depth cues to provide a pseudo-three-dimensional perspective. This enhanced depth perception is helpful for medical professionals to accurately navigate and identify anatomical structures during medical procedures.

As shown in FIG. 9, a flowchart is provided detailing the real-time anatomical highlighting process 900. It highlights the sequence of steps from the initial video feed input 902 to AI anatomical recognition 904 to highlighting key structures 906 to the final stage where key anatomical structures are visually enhanced in the feed 908. This real-time highlighting aids medical professionals in quickly identifying critical areas, improving procedural accuracy and efficiency.

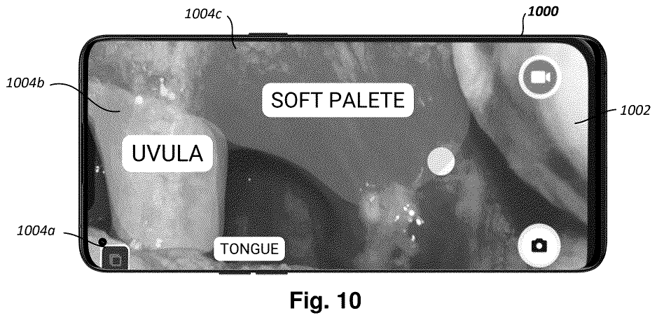

One example of the output of process 900 is shown in FIG. 10. In this figure, a mockup of the user interface 1000 is shown, demonstrating how the enhanced video feed, complete with depth perception cues and anatomical highlights, may be presented to the medical professionals. Specifically, a real-time feed 1002 of captured video or images from a laryngoscope may be presented on user interface 1000 and certain anatomical features may be marked and called out. In this example, three anatomical features 1004a,1004b,1004c are presented to the user, corresponding to the tongue, the uvula and the soft palate of the patient. In this example, the three features are highlighted or colored in yellow, green and blue, respectively, but the opacity of the markings are adjusted so that the underlying structures or features can be seen through the colored overlay (e.g., the color may have an opacity of 5%, 10%, 15%, 20%, 30%, 40%, 50%, 60%, 70%, 80%, 90% or more). Alternatively, the colored overlay may be completely opaque and can replace parts of the image. In some examples, the detected anatomical features may be replaced with an icon or simple illustration, or the outline of the anatomical feature may be shaped, colored or bound within a border. In presenting the information to the patient in this manner, the interface design focuses on clarity, ease of navigation, and the ability to customize the level of enhancement based on specific procedural requirements.

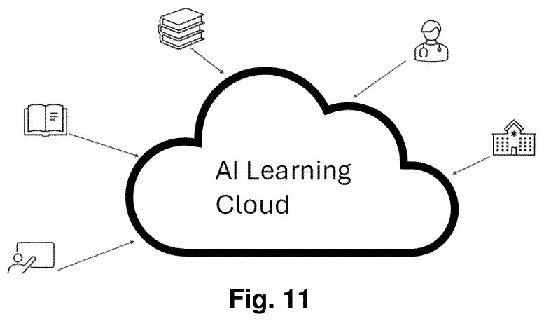

The continuous learning and improvement aspect of the medical system is depicted in FIG. 11. This diagram illustrates the integration of various data sources into the system, and specifically the AI learning cloud database. In some examples, the data sources may include one or more of direct feedback from medical professionals, literature (e.g., textbooks and journal articles), educational sources, patient outcomes, and a broad spectrum of procedural videos. This continuous learning mechanism allows the AI algorithm to evolve, improving its accuracy and effectiveness over time.

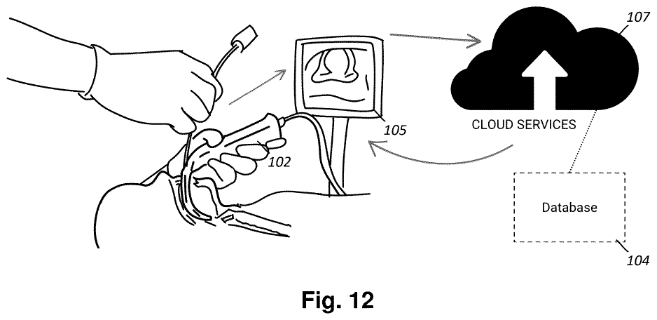

FIG. 12 illustrates the schematic of the system's integration in a typical medical environment. It displays the connections between the medical imaging device or procedural element 102, the interface unit 105, alternatively the external processing unit 106, and the AI processing unit within the interface unit 105. Also depicted is the cloud-based service 107, which facilitates the AI's continuous learning and updates from a database 104. The diagram emphasizes the flow of data and control signals between these components, highlighting how the interface unit 105 serves as the central hub for receiving, processing, and transmitting information.

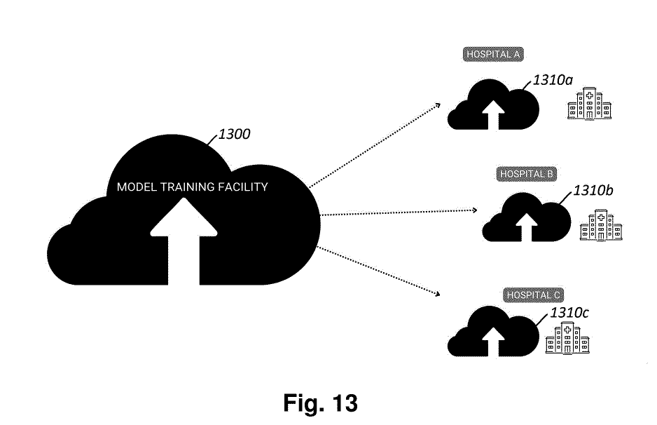

In some embodiments, a cloud-based model training and update facility is an integral component of the system, as detailed in FIG. 13. This figure demonstrates how the system's model training facility 1300 aggregates, processes, and utilizes extensive data sets from multiple hospitals 1310a, 1310b, 1310c to continually enhance the AI model's capabilities. The data sets from these hospitals include a variety of metrics such as the number of procedures, types of procedures, experience level of the primary physician performing the procedure, length of the procedure, relative success of the procedure, patient demographics (age, gender, medical history), anatomical area of focus, procedural complications, image quality and clarity, frequency of use of specific procedural elements, patient recovery data, interventional outcomes, equipment used during procedures, and environmental variables (lighting, operating room setup). These parameters are meticulously analyzed to refine the AI model, ensuring a comprehensive learning process that encompasses diverse medical scenarios and patient profiles. Thus, the cloud infrastructure may facilitate scalable AI model training and continuous updates. The model training may include a model training facility that aggregates procedural data from multiple sources, including various hospitals and clinical settings. The facility may employ distributed computing resources to train and refine AI models based on diverse procedural scenarios. The model training may also include a data processing pipeline that processes raw procedural data, including video feeds, procedural parameters, and clinician feedback, to enhance AI model accuracy and adaptability. The model may also include model deployment and update mechanism that allows for updated AI models to be seamlessly integrated into the external processing system and edge deployment hardware, maintaining system performance and reliability.

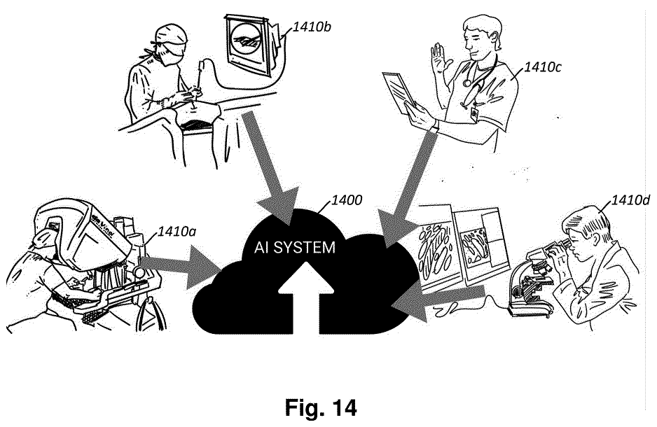

Lastly, FIG. 14 provides a comprehensive illustration of the wide-ranging applicability of the system 1400 across various medical procedures and settings. This diagram showcases the system's versatility and integration capabilities with different medical devices and environments. It features specific applications such as robotic surgical systems 1410a, where the system enhances precision and depth perception in minimally invasive surgeries; laparoscopic procedures 1410b, demonstrating the system's utility in providing enhanced visualization for internal examinations and surgeries; telehealth and remote diagnostics 1410c, illustrating the system's adaptability in providing high-quality imaging and diagnostic support in remote healthcare delivery; and research activities 1410d, indicating the system's role in facilitating detailed medical research through improved imaging and data analysis capabilities. The diagram emphasizes the system's potential to revolutionize a variety of medical processes by providing AI-enhanced imaging, thereby contributing significantly to advancements in healthcare technology and patient care.

In some examples, a method is provided for AI-driven assistance in critical medical procedures. The method includes providing a procedural element configured for insertion into a body, equipped with a high-resolution monocular camera for capturing real-time video feeds. During a medical procedure, the procedural element is inserted into the body, and the camera captures the real-time video feed. This feed is transmitted via a wired communication interface to an external processing system, where artificial intelligence (AI) algorithms process the feed to perform real-time anatomical tagging, provide guidance feedback, and execute assimilated scenario testing. The AI-driven outputs, including anatomical tags and guidance feedback, are displayed to clinicians through an interactive user interface.

In some examples, the artificial intelligence algorithms incorporate semantic segmentation and object detection techniques for anatomical tagging. The system's real-time guidance functionality utilizes predictive analytics to dynamically adjust the positioning and orientation of the procedural element. Furthermore, augmented reality (AR) simulations are used to test and validate system performance in complex scenarios, including challenges such as fluid obstructions, low-light conditions, and anatomical variations. Machine learning techniques within the external processing system may continuously refine anatomical tagging and guidance feedback based on procedural data and clinician input.

In some examples, the system supports advanced customization and integration capabilities. The interactive user interface allows clinicians to tailor anatomical tagging parameters and guidance feedback settings to specific procedural requirements. Additionally, procedural data, including video feeds and AI-driven assistance outputs, can be stored in a cloud-based storage system for further analysis and model training. The external processing system includes a labeling tool with an inference server hosting pre-trained AI models for rapid and accurate annotation of anatomical structures, ensuring efficient data labeling and improved dataset accuracy.

In some examples, a comprehensive system is provided for AI-driven assistance, comprising edge deployment hardware for real-time AI inference with minimal latency. The system features a procedural element compatible with various medical devices such as endoscopes, bronchoscopes, cystoscopes, and video laryngoscopes, enabling its use across multiple specialties. A cloud-based infrastructure supports scalable AI model training and updates, adapting to evolving medical practices and procedural insights. A computer program product with computer-readable instructions stored on a non-transitory medium enables the described method, ensuring robust, adaptable, and efficient AI-driven support for critical medical procedures.

Thus, the elements illustrated in the figures with the textual descriptions provide a comprehensive understanding of the medical system. The system represents a significant advancement in medical imaging technology, offering an innovative, AI-driven approach to enhancing the safety, accuracy, and efficiency of various medical procedures. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of enhancing a monocular video feed, comprising:
inserting a procedural element into a body, the procedural element having a camera;
capturing the monocular video feed, with the camera, of at least one anatomical structure within the body; and
sending the monocular video feed to an external system having a processor and a memory, the external system being configured to (1) process the monocular video feed, and (2) generate an enhanced video feed, the enhanced video feed including a real-time, three-dimensional perspective of the at least one anatomical structure.

2. The method of claim 1, wherein the external system is configured to process the monocular video feed using an artificial intelligence algorithm.

3. The method of claim 1, wherein the external system is configured to process the monocular video feed using machine learning algorithms capable of identifying and visually enhancing certain anatomical structures from the monocular video feed.

4. The method of claim 1, wherein the external system is configured to enhance depth perception according to adjustable user preferences and specific procedural requirements.

5. The method of claim 1, wherein the external system is capable of at least one of color enhancement and contour outlining of particular anatomical structures to make the particular anatomical structures more visually prominent.

6. The method of claim 1, further comprising providing a semi-transparent color overlay on a particular anatomical structure and displaying an image that includes the semi-transparent color overlay disposed over the particular anatomical structure.

7. The method of claim 1, further comprising continuously updating the external system by analyzing a diverse set of procedural videos and incorporating feedback for algorithm refinement within the external system.

8. The method of claim 7, wherein continuously updating the external system comprise reinforcement learning techniques and periodic updates to AI models based on new data and insights.

9. The method of claim 7, further comprising integrating the external system with existing healthcare IT infrastructures and sharing information with electronic health records and diagnostic imaging systems.

10. The method of claim 7, further comprising providing real-time feedback and guidance through a user-friendly interface.

11. The method of claim 1, further comprising providing a cloud-based framework for model training and updates, wherein the framework enables scalable learning and adaptation to new technologies and medical practices.

12. The method of claim 1, further comprising operating the external system using a multimodal interaction interface that supports touch, voice commands, and haptic feedback.

13. The method of claim 1, wherein the external system is adaptable to different healthcare settings and support multiple languages.

14. The method of claim 1, wherein the external system features interactive learning interfaces and customizable learning paths for medical professionals.

15. The method of claim 1, further comprising an anatomical highlighting algorithm that adapts and improves its accuracy through continuous learning based on a vast array of procedural videos.

16. The method of claim 1, further comprising providing a feedback mechanism to allow medical professionals to provide input and annotations to aid an algorithm's learning and accuracy.

17. The method of claim 1, wherein the procedural element is capable of being used in an endoscopy, a bronchoscopy, a cystoscopy, and a video laryngoscopy.

18. A non-transitory computer readable medium including instructions that, when executed by at least one processor, cause the at least one processor to perform a method for enhancing a monocular video feed, the method comprising:
receiving capturing the monocular video feed of at least one anatomical structure within the body from a procedural element having a camera;
processing the monocular video feed, and
generating an enhanced video feed, the enhanced video feed including a real-time, three-dimensional perspective of the at least one anatomical structure.
